(19) United States
(12) Patent Application Publication
(10) Pub. No.: US 2005/0209178 A1
(43) Pub. Date: Sep. 22, 2005

(54) Methods and compositions for the treatment of obesity

(76) Inventor: Stefan Pulst, Los Angeles, CA (US)

Correspondence Address: JONES DAY, 555 SOUTH FLOWER STREET FIFTIETH FLOOR, LOS ANGELES, CA, 90071, US

(21) Appl. No.: 10/802,228

(22) Filed: Mar. 16, 2004

(51) Int. Cl. A61K 48/00; C12N 15/09

(52) U.S. Cl. 514/44; 435/320.1

(57) ABSTRACT

The present invention provides novel compositions containing a SCA-2 therapeutic element, which is involved in the regulation normal body weight and may be administered for the prevention of an abnormal accumulation of body fat. In particular the compositions and methods of the present invention may be used for the prevention or reversal of obesity.

### FIELD OF THE INVENTION

The present invention relates to the fields of molecular biology, gene- and protein-based therapeutics, and the treatment of obesity. Specifically, the present invention is based on the determination that a novel gene, SCA-2, is required for the maintenance of normal body weight and that loss of function of the gene leads to obesity. Thus, the present invention provides compositions and methods that are useful for the maintenance of normal body weight and the treatment of obesity.

### BACKGROUND OF THE INVENTION

Various publications or patents are referred to throughout this application or at the end of this specification to describe the state of the art to which the invention pertains. Each of these publications or patents is incorporated by reference herein. Citations of scientific publications are set forth in the text or at the end of the specification.

Spinocerebellar Ataxia 2 is a neurodegenerative disease caused by the abnormal expansion of an unstable CAG repeat in the SCA-2 gene. The protein product of the SCA-2 gene is also known as ataxin-2, but will herein be referred to as SCA-2, for the sake of simplicity. Patients afflicted with spinocerebellar ataxia 2 show a variety of clinical symptoms ranging from ataxia and other movement disorders to neuropathy and dementia. The severity and onset of the symptoms has been correlated with the size of the abnormal CAG repeat expansion in the SCA-2 gene. SCA-2 is a member of a novel protein family with putative RNA-binding functions that are evolutionarily conserved. A surprising finding was that the deletion of SCA-2 by knocking out the gene causes the disruption of normal body weight maintenance and leads to the onset of obesity. The present invention makes use of the requirement for SCA-2 gene function in the maintenance of normal body weight by providing compositions and methods for the prevention and/or reversal of obesity.

### SUMMARY OF THE INVENTION

The present invention provides novel compositions containing an SCA-2 therapeutic, which is involved in the regulation of normal body weight, and may be administered for the prevention or reversal of obesity in individuals.

In one embodiment, the present invention provides an isolated polynucleotide encoding an SCA-2 protein. Preferably, the polynucleotide comprises the sequence of: SEQ ID NO:1, with up to 30% conservative substitutions; an allelic variant of SEQ ID NO:1; a sequence hybridizing with SEQ ID NO:1 or its complement under stringent hybridization and washing conditions.

Another embodiment of the instant invention features a recombinant SCA-2 DNA or RNA molecule comprising a vector having an insert that includes part or all of an SCA-2 polynucleotide sequence. The invention also features a vector containing an SCA-2 polypeptide. Pharmaceutical compositions containing a biologically effective amount of the SCA-2 polynucleotide, protein and/or protein fragments with acceptable carriers are also provided.

Hence, in another, particular embodiment, the present invention may be implicated in the treatment of obesity. Therefore, the invention relates to compositions and methods for treating morbid conditions associated with an abnormal increase in body fat, by administering an SCA-2 gene or protein to prevent or reverse obesity. Specifically, a method of treating abnormal body fat accumulation involving the administration of a pharmaceutical composition containing a biologically effective amount of an SCA-2 polynucleotide or polypeptide along with an acceptable carrier, is provided.

Specifically, in a particular embodiment, the invention relates to the administration of vectors for the delivery of an SCA-2 therapeutic to a cell for the treatment of obesity, wherein the vector contains an expression cassette encoding the SCA-2 therapeutic. The SCA-2 therapeutic can be an SCA-2 polynucleotide, an SCA-2 protein, or an SCA-2 protein fragment. The expression cassette may contain one or more of the following elements: a host cell origin of replication, a suitable promoter operably linked to a heterologous genetic element, an internal ribosome entry site, splice donor site, splice acceptor site, a suitable enhancer, PPT track, heterologous genetic element, a reporter gene, and/or an appropriate termination sequence. One or more of these vectors, containing an SCA-2 therapeutic, may be introduced into an appropriate cell by a variety of means, including in vivo, in vitro or ex vivo transduction or transfection using an appropriate expression system.

### BRIEF DESCRIPTION OF THE DRAWINGS

The invention is best understood from the following detailed description when read in connection with the accompanying drawings, in which:

FIG. 1 shows the SCA-2 protein product and its domains.

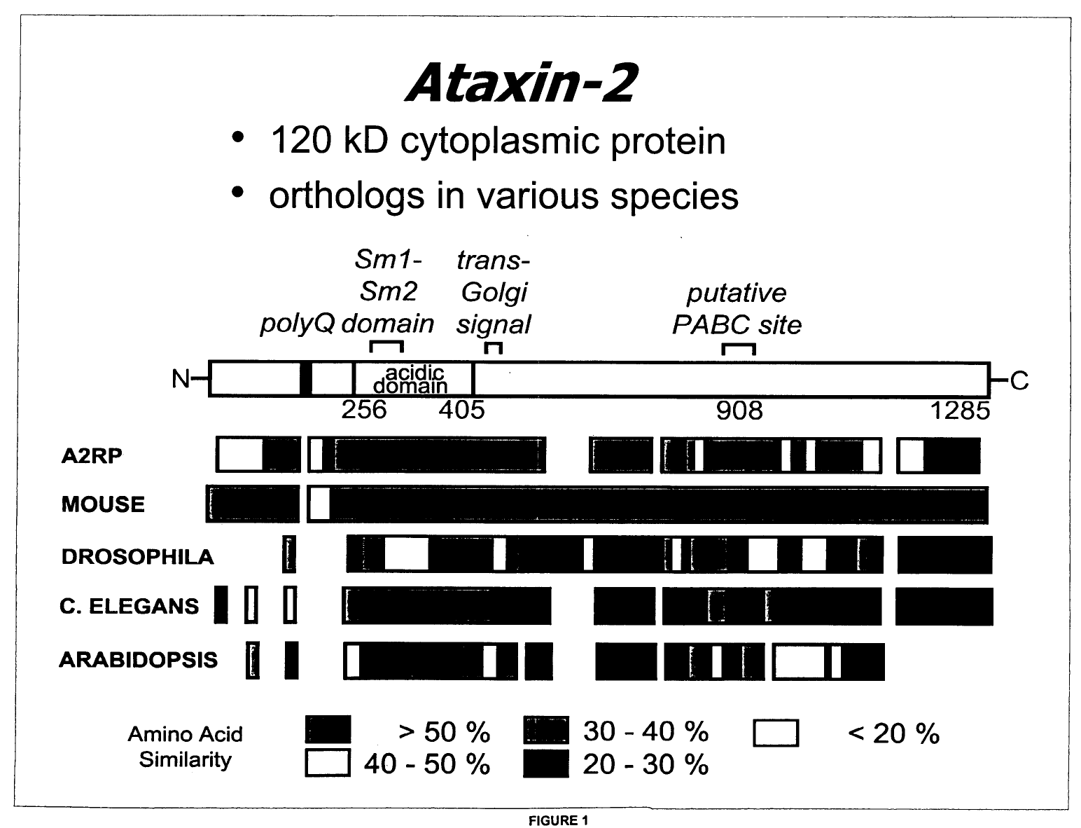

FIG. 2 shows the increase in body weight gain of SCA-2 knockout mice compared to wild-type mice.

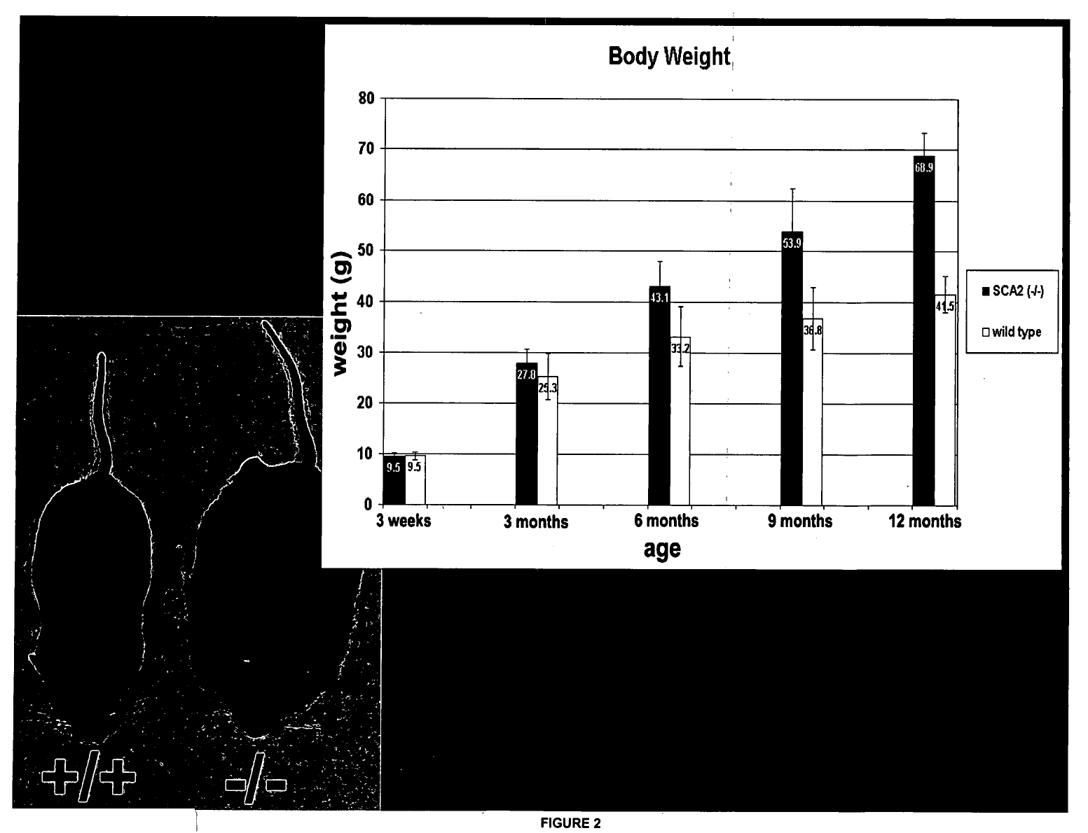

FIG. 3 shows a body weight comparison between SCA-2 knockout mice, SCA-2 (+/−) hemizygotes, and wild type mice (+/+).

FIG. 4 shows the average daily food intake for the three genotypes under unrestricted conditions.

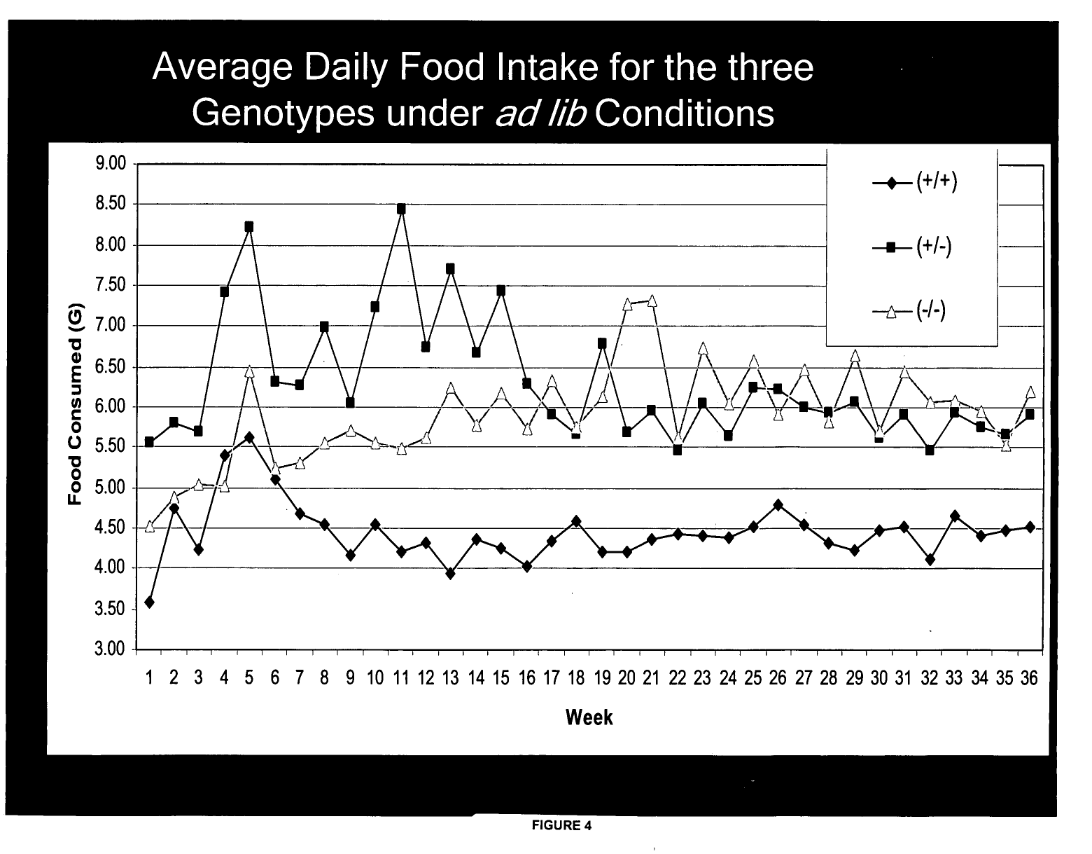

FIG. 5 shows the effect of a restricted diet on the body weight for each of the three genotypes.

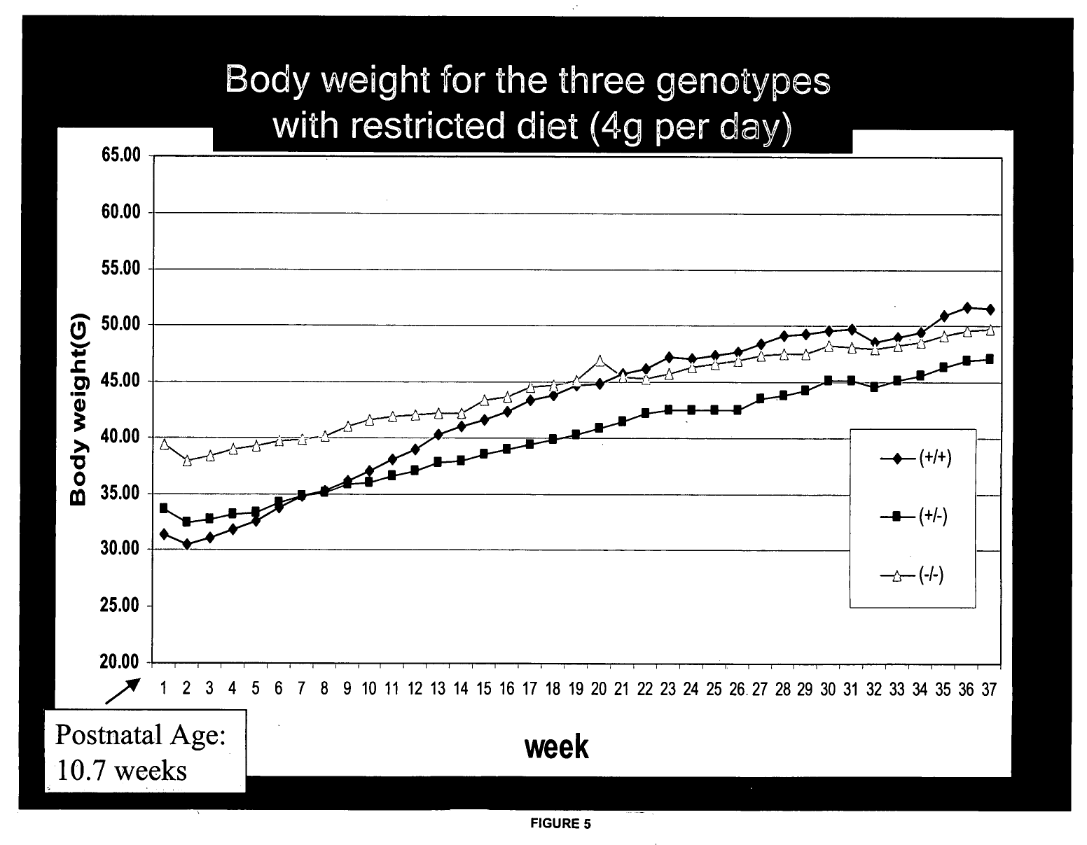

FIG. 6 shows the average daily food intake for the three genotypes under restricted conditions.

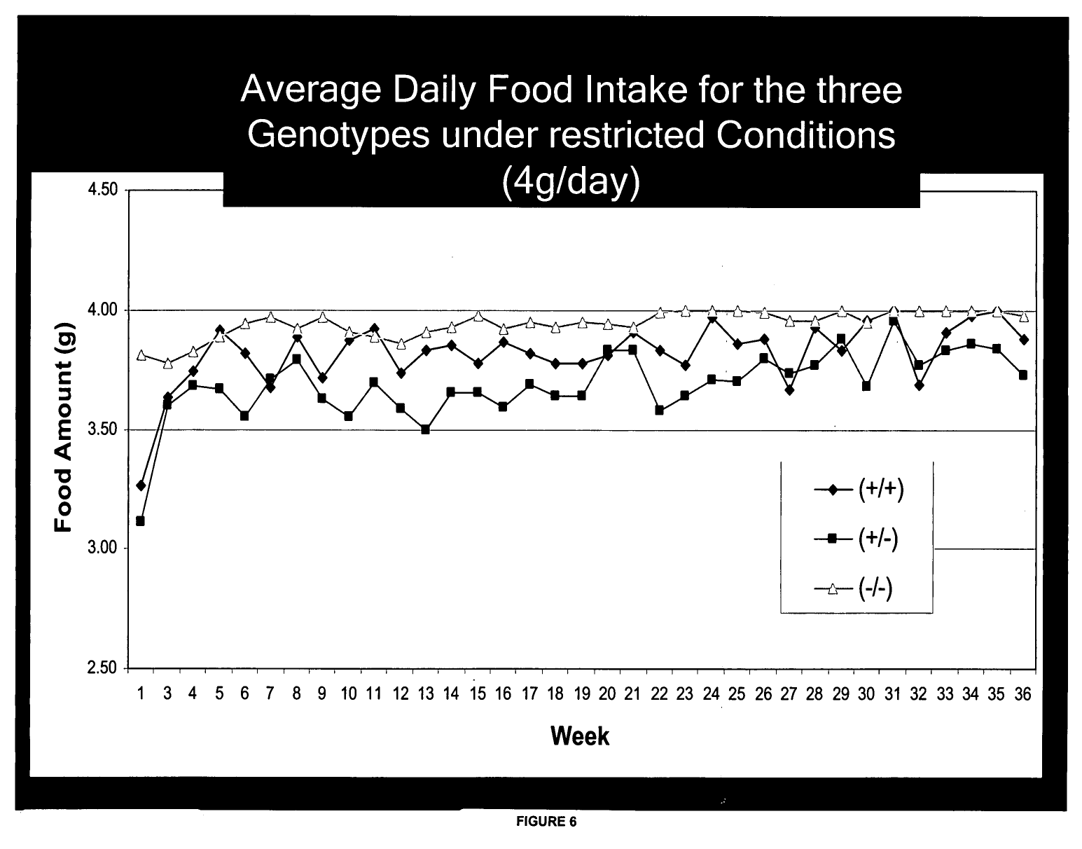

FIG. 7 shows that SCA-2 knockout and hemizygous mice are heavier than wild type mice regardless of diet restriction.

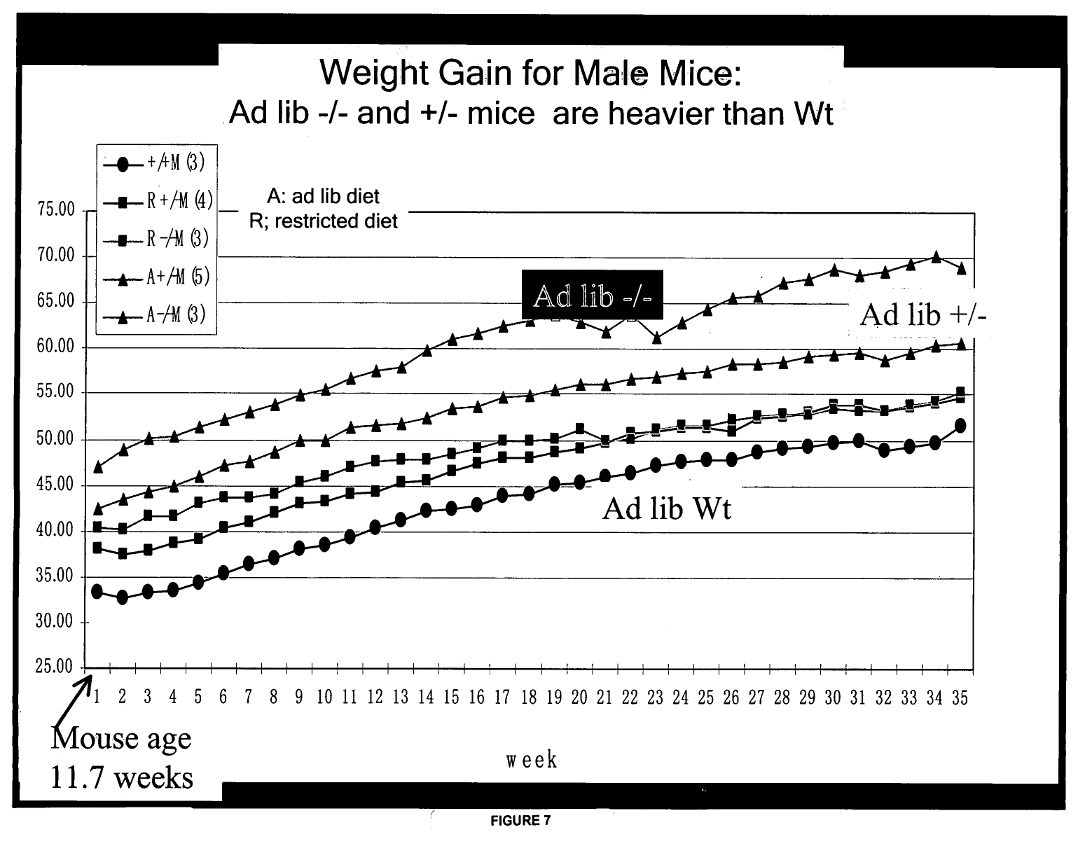

### DETAILED DESCRIPTION OF THE INVENTION

Introduction

In light of the recent work by the Applicant, SCA-2 has been identified as a gene involved in the regulation of normal body weight the absence of which leads to obesity. Obesity, defined as a state of excess of adipose tissue mass, has become a serious public health concern in most industrialized nations. The most widely used method to gauge obesity is the body mass index (BMI), which is calculated by dividing an individual's weight in kilograms by the square of his or her height in centimeters. While a BMI of 30 is most commonly used as a threshold for obesity in both men and women, epidemiologic studies. have shown that all-cause, metabolic, and cardiovascular morbidity begin to rise when BMIs are above 25. Obesity has major adverse health effects, including insulin resistance and type II diabetes, reproductive disorders, cardiovascular disorders and hypertension, pulmonary disease, reproductive disorders, bone, joint and cutaneous disease, and cancer. The prevalence of obesity in the American population is on the rise and recent survey data suggests that more than half of the adult population and an alarmingly high percentage of children have a BMI exceeding 25, putting them at risk for significant health consequences.

Obesity is a multifactorial condition that is influenced by a complex interplay between endocrine and neural components regulating appetite, metabolic rate, and physical activity. While some of the genes involved in the regulation of body weight and adiposity have been elucidated, the majority of genes and gene products that form part of the complex network of body weight maintenance remain to be identified. The present invention represents a major step toward solving the puzzle of obesity and provides novel compositions and methods for the treatment thereof. In particular, the present invention provides methods and compositions believed to be particularly useful in the prevention and treatment of stress-induced obesity. Although specific embodiments of the present invention will now be described, it should be understood that such embodiments are by way of example only and merely illustrative of but a small number of the many possible specific embodiments that can represent applications of the principles of the present invention. Various changes and modifications obvious to one skilled in the art to which the present invention pertains are deemed to be within the spirit, scope and contemplation of the present invention as further defined in the appended claims.

Definitions

Various terms relating to the biological molecules of the present invention are used throughout the specification and claims.

By “obesity” is meant the abnormal accumulation of body fat, also referred to as adipose tissue, above a medically relevant threshold, such as a BMI exceeding 25.

“SCA-2” refers generally to an SCA-2 polypeptide that has been shown to be involved in obesity, in accordance with the present invention, which is described in detail herein above and throughout the specification.

“SCA-2 activity or SCA-2 polypeptide activity” or “biological activity of the SCA-2 protein or SCA-2 polypeptide” refers to the metabolic or physiologic function of said SCA-2 including similar activities or improved activities or these activities with decreased undesirable side effects. In particular, the SCA-2 polynucleotide encodes a protein that has been shown to be involved in the regulation of normal body weight in that its absence leads to obesity.

“SCA-2 gene” refers to a polynucleotide in accordance with the present invention, which encodes an SCA-2 polypeptide.

An “SCA-2 therapeutic” refers to a therapeutically effective amount of an SCA-2 related genetic sequence such as, but not limited to the polynucleotide sequence, and SCA-2 peptide, protein or protein fragment.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

“Polynucleotide” generally refers to any polyribonucleotide or polydeoxyribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA.

The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the gene-encoded amino acids. “Polypeptides” include amino acid sequences modified either by natural processes, such as posttranslational processing, or by chemical modification techniques that are well known in the art. Such modifications are well described in basic texts and in more detailed monographs; as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched circular polypeptides may result from posttranslation natural processes or may be made by synthetic methods.

Modifications include acetylation, acylation, ADP-ribosylation, amidation, covalent attachment of various moiety groups, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross links, formation of cystine, formation of pyroglutamate, formylation, gamma-carboxylation, glycosylation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Proteins—Structure And Molecular Properties, 2nd Ed., T. E. Creighton, W. H. Freeman and Company, New York, 1993 and Wold, F., “Posttranslational Protein Modifications: Perspectives and Prospects, pgs. 1-12 in “Posttranslational Covalent Modification Of Proteins”, B, C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical.

A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “substantially the same” refers to nucleic acid or amino acid sequences having sequence variations that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

With respect to oligonucleotide constructs, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide construct with a substantially complementary sequence contained within a single-stranded DNA or RNA molecule of the invention, to the substantial exclusion of hybridization of the oligonucleotide with single-stranded nucleic acids of non-complementary sequence.

The term “substantially pure” refers to a “preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate to the compound of interest (e.g. chromatographic methods, agarose or polyacrylamide gel electrophoresis, HPLC analysis, and the like).

The term “expression cassette” refers to a nucleotide sequence that contains at least one coding sequence along with sequence elements that direct the initiation and termination of transcription. An expression cassette may include additional sequences, including, but not limited to promoters, enhancers, sequences involved in post-transcriptional or post-translational processes, as well as appropriate terminator sequences.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers and regulators) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a vector for transforming a cell, in vitro or in vivo. This term may be used interchangeably with the term “transforming DNA”. Such a nucleic acid construct may contain a coding sequence for a gene product of interest, along with a selectable marker gene and/or a reporter gene.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The term “DNA construct”, as defined above, is also used to refer to a heterologous region, particularly one constructed for use in transformation of a cell. A cell has been “transformed” or “transfected” or “transduced” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

The term “in vivo delivery” involves the use of any gene delivery system, such as viral- and liposome-mediated transformation for the delivery and introduction of a therapeutic agent to the cells of a subject while they remain in the subject. Such therapeutic elements may include, for example, DNA, cDNA, RNA, and antisense polynucleotide sequences.

As used herein, the term “transduction,” is used to describe the delivery of DNA to eukaryotic cells using viral mediated delivery systems, such as, adenoviral, AAV, retroviral, or plasmid delivery gene transfer methods. Preferably the viral mediated delivery system is targeted specifically to the cell, wherein delivery is sought. The production of targeted delivery systems is well known and practiced in the recombinant arts. A number of methods for delivering therapeutic formulations, including DNA expression constructs (as described further below), into eukaryotic cells are known to those skilled in the art. In light of the present disclosure, the skilled artisan will be able to deliver the therapeutic elements of the present invention to cells in many different but effective ways. Naturally, different viral host ranges will dictate the virus chosen for gene transfer.

In vitro gene delivery” refers to a variety of methods for introducing exogenous DNA into a cell that has been removed form its host environment.

As used herein the term, “transfection” is used to describe the delivery and introduction of a therapeutic agent to a cell using non-viral mediated means, these methods include, e.g., calcium phosphate- or dextran sulfate-mediated transfection; electroporation; glass projectile targeting; and the like. These methods are known to those of skill in the art, with the exact compositions and execution being apparent in light of the present disclosure.

“Ex vivo gene delivery” refers to the procedure wherein appropriate cells are removed form the host organism, transformed, transduced or transfected in accordance with the teachings of the present invention, and replaced back into the host organism, for the purpose of therapeutic restoration and/or prevention.

“Delivery of a therapeutic element or agent” may be carried out through a variety of means, such as by using parenteral delivery methods such as intravenous and subcutaneous injection, and the like. Such methods are known to those of skill in the art of drug delivery, and are further described herein in the sections regarding pharmaceutical preparations and treatment. Compositions, include pharmaceutical formulations, comprising an SCA-2 gene, protein, or antisense polynucleotide sequence that may be delivered. In such compositions, the SCA-2 may be in the form of a DNA segment, plasmid, recombinant vector or recombinant virus that is capable of expressing an SCA-2 protein in a cell. These compositions, including those comprising a recombinant viral gene delivery system, such as an adenovirus particle, may be formulated for in vivo administration by dispersion in a pharmacologically acceptable solution or buffer. Preferred pharmacologically acceptable solutions include neutral saline solutions buffered with phosphate, lactate, Tris and the like.

The term “contacted” when applied to a cell is used herein to describe the process by which an SCA-2 genetic element, such as a gene, protein or antisense sequence is delivered to a target cell or is placed in direct proximity with the target cell. This delivery may be in vitro or in vivo and may involve the use of a recombinant vector system. Any method may be used to contact a cell with the SCA-2 associated protein or nucleotide sequence, so long as the method results in increased levels of functional SCA-2 protein within the cell. This includes both the direct delivery of an SCA-2 protein to the cell and the delivery of a gene or DNA segment that encodes SCA-2, which gene sequence will direct the expression and production of SCA-2 within the cell. Since protein delivery is subject to drawbacks, such as degradation and low cellular uptake, it is contemplated that the use of a recombinant vector that expresses an SCA-2 protein will be of particular advantage for delivery.

By a “therapeutically effective amount” is meant an amount of the polynucleotide or protein of, or fragment thereof, that when administered to a subject is effective to bring about a desired effect (e.g., a decrease of body fat) within the subject.

Polynucleotides

The present invention provides a novel composition containing a therapeutic SCA-2 genetic element, such as a gene or protein, which acts to inhibit obesity. The present invention concerns compositions and methods for treating various diseases associated with an unhealthy increase in body fat. The invention is based firstly on the inventor's discovery that the disruption of SCA-2 causes the onset of obesity. Thus, SCA-2 plays a role in maintaining normal body weight, and may be useful in the treatment of obesity.

As described in more detail infra, the SCA-2 gene can be cloned into different expression vector constructs. The human SCA-2 cDNA is set out in SEQ ID NO:1. The SCA-2 polynucleotides of the present invention include isolated polynucleotides encoding SCA-2 proteins, polypeptides and/or fragments, and polynucleotides closely related thereto. More specifically, SCA-2 polynucleotides of the invention include a polynucleotide comprising the human nucleotide sequences contained in SEQ ID NO:1 encoding a SCA-2 polypeptide of SEQ ID NO:2, and polynucleotides having the particular sequence of SEQ ID NO:1.

SCA-2 polynucleotides further include a polynucleotide comprising a nucleotide sequence that has at least 70% identity over its entire length to a nucleotide sequence encoding the SCA-2 polypeptide of SEQ ID NO:2, and a polynucleotide comprising a nucleotide sequence that is at least 70% identical to that of SEQ ID NO:1, over its entire length. In this regard, polynucleotides with at least 70% are preferred, more preferably at least 80% even more preferably at least 90% identity, yet more preferably at least 95% identity, 97% are highly preferred and those with at least 98-99% are most highly preferred, with at least 99% being the most preferred.

The present invention includes polynucleotides encoding polypeptides which have at least 70% identity, preferably at least 80% identity, more preferably at least 90% identity, yet more preferably at least 95% identity, even more preferably at least 97-99% identity, to the amino acid sequence of SEQ ID NO:2, over the entire length of the recited amino acid sequences. The nucleotide sequences encoding the SCA-2 polypeptide of SEQ ID NO:2 may be identical to the polypeptide encoding sequence contained in SEQ ID NO:1, or it may be a sequence, which as a result of the redundancy (degeneracy) of the genetic code, also encodes the polypeptide of SEQ ID NO:2. Also included under SCA-2 polynucleotides are nucleotide sequences that code for polynucleotides that are complementary to such SCA-2 polynucleotides, such as anti-sense SCA-2 polynucleotide sequences.

SCA-2 polynucleotides of the present invention may be prepared by two general methods: (1) they may be synthesized from appropriate nucleotide triphosphates, or (2) they may be isolated from biological sources. Both methods utilize protocols well known in the art. The availability of nucleotide sequence information, such as the cDNA having SEQ ID NO:1, enables preparation of an isolated nucleic acid molecule of the invention by oligonucleotide synthesis.

Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC). Long, double-stranded polynucleotides, must be synthesized in stages, due to the size limitations inherent in current oligonucleotide synthetic methods. Thus, for example, a long double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire long double-stranded molecule. A synthetic DNA molecule so constructed may then be cloned and amplified in an appropriate vector. In this respect, the polynucleotide may also contain non-coding 5′ and 3′ sequences, such as transcribed, non-translated sequences, splicing and polyadenylation signals, ribosome binding sites and sequences that stabilize mRNA.

SCA-2 genes may be isolated from appropriate biological sources using methods known in the art. In the exemplary embodiment of the invention, SCA-2 may be isolated from cDNA libraries of human brain as described in Pulst et al., (1996) Nature Genetics 14:3, 269-276. A preferred means for isolating SCA-2 genes is PCR amplification using genomic or cDNA templates and SCA-2 specific primers. Genomic and cDNA libraries are commercially available, such as those sold by Sigma, and can also be made by procedures well known in the art. In positions of degeneracy where more than one nucleic acid residue could be used to encode the appropriate amino acid residue, all the appropriate nucleic acid residues may be incorporated to create a mixed oligonucleotide population, or a neutral base such as inosine may be used. The strategy of oligonucleotide design is well known in the art. Nucleic acids of the present invention may be maintained as DNA in any convenient cloning vector. In a preferred embodiment, clones are maintained in plasmid cloning/expression vector, such as pBluescript (Stratagene, La Jolla, Calif.), that is propagated in a suitable E. coli host cell.

Hence, in one particular aspect, the present invention provides novel compositions containing a SCA-2 therapeutic element, which is involved in the regulation of normal body weight, and may be administered for the prevention or reversal of an abnormal body fat accumulation in individuals. More particularly, the invention pertains to novel compositions containing a SCA-2 polynucleotide sequence that may be used for the prevention or reversal of stress-induced weight gain or obesity in susceptible individuals. More particularly still, the polynucleotide compositions and methods of the present invention may be useful in the reversal or prevention of high fat diet induced obesity in individuals.

Specifically, according to one aspect, this invention depends on an isolated polynucleotide encoding a SCA-2 protein. Preferably, the polynucleotide comprises the sequence of: SEQ ID NO:1, with up to 30% conservative substitutions; an allelic variant of SEQ ID NO:1; a sequence hybridizing with SEQ ID NO:1 or its complement under stringent hybridization and washing conditions. In a particular preferred embodiment, a pharmaceutical composition containing a therapeutically effective amount of an SCA-2 polynucleotide along with a biologically acceptable carrier is provided, whereby the administration of said composition will be useful for the prevention and/or treatment of obesity.

Polypeptides

In one aspect, the present invention relates to SCA-2 polypeptides, SCA-2 proteins, or therapeutically active fragments thereof. The SCA-2 polypeptides include the polypeptide of SEQ ID NO:2; as well as polypeptides comprising the amino acid sequence of SEQ ID NO:2; and polypeptides comprising the amino acid sequences that have at least 70% identity to that of SEQ ID NO:2, over its entire length. Preferably SCA-2 polypeptides or proteins exhibit at least one biological activity of SCA-2, specifically the ability to regulate body fat accumulation and prevent obesity. The present invention further provides for a polypeptide that comprises an amino acid sequence that has at least 80% identity, more preferably at least 90% identity, yet more preferably at least 95% identity, most preferably at least 97-99% identity, to that of SEQ ID NO:2 over the entire length of SEQ ID NO:2.

The SCA-2 polypeptides may be in the form of the “mature” protein or may be a part of a larger protein, such as a fusion protein. It is often advantageous to include an additional amino acid sequence which contains secretory or leader sequences, pre/pro-sequences, sequences which aid in purification such as multiple histidine residues, or an additional sequence for stability during recombinant production.

Fragments of the SCA-2 proteins are also included in the invention. A fragment is a polypeptide having an amino acid sequence that entirely is the same as part, but not all, of the amino acid sequence of the aforementioned SCA-2 polypeptides. Preferred fragments include, for example, truncation polypeptides having the amino acid sequence of SCA-2 polypeptides, except for deletion of a continuous series of residues that includes the amino terminus, or a continuous series of residues that includes the carboxyl terminus or deletion of two continuous series of residues, one including the amino terminus and one including the carboxyl terminus, but having the same functionality as the endogenous SCA-2 protein, namely, the ability to regulate body fat accumulation and to prevent or reverse obesity. Other preferred fragments are biologically active fragments. Biologically active fragments are those that mediate SCA-2 activity, including those with a similar activity or an improved activity, or with a decreased undesirable activity. Also included are those that are antigenic or immunogenic in an animal, especially in a human. Preferably, all of these polypeptide fragments retain the biological activity of the SCA-2. Variants of the defined sequence and fragments also form part of the present invention. Preferred variants are those that vary from the referents by conservative amino acid substitutions.

The SCA-2 proteins and polypeptides of the invention can be prepared in any suitable manner or purchased, recombinantly, from commercial sources. If produced in situ, the polypeptides may be purified from appropriate sources, e.g., appropriate vertebrate cells e.g., mammalian cells, for instance cells from human, mouse, bovine or rat. Alternatively, the availability of nucleic acid molecules encoding the polypeptides enables production of the proteins using in vitro expression methods well known in the art. For example, a cDNA or gene may be cloned into an appropriate in vitro transcription vector, for in vitro transcription, followed by cell-free translation in a suitable cell-free translation system. In vitro transcription and translation systems are commercially available, e.g., from Promega Biotech, Madison, Wis., or BRL, Rockville, Md. While in vitro transcription and translation is not the method of choice for preparing large quantities of the protein, it is ideal for preparing small amounts of native or mutant proteins for research purposes, particularly since it allows the incorporation of radioactive nucleotides.

Larger quantities of SCA-2 encoded polypeptide may be produced by expression in a suitable prokaryotic or eukaryotic system. For example, part or all of a DNA molecule, such as SEQ ID NO:1 may be inserted into a plasmid vector adapted for expression in a bacterial cell (such as E. coli) or a yeast cell (such as Saccharomyces cerevisiae). Such vectors comprise the regulatory elements necessary for expression of the DNA in the host cell, positioned in such a manner as to permit expression of the DNA into the host cell. Such regulatory elements required for expression include appropriate origins of replication, promoter sequences, transcription initiation sequences and optionally, enhancer or termination sequences. Secretion signals may be used to facilitate purification of the resulting protein. An appropriate secretion coding sequence for the secretion of the peptide is operably linked to the 5′ end of the coding sequence for the protein, and this hybrid nucleic acid molecule is inserted into a plasmid adapted to express the protein in the host cell of choice. Plasmids specifically designed to express and secrete foreign proteins are available from commercial sources. For example, if expression and secretion is desired in E. coli, commonly used plasmids include pTrcPPA (Pharmacia); pPROK-C and pKK233-2 (Clontech); and pNH8a, pNH16a, pcDNAII and pAX (Stratagene), among others.

The SCA-2 proteins produced by in vitro transcription and translation or by gene expression in a recombinant prokaryotic or eukaryotic system may be purified according to methods known in the art. Recombinant proteins can be purified by affinity separation, such as by immunological interaction with antibodies that bind specifically to the recombinant protein or fusion proteins such as His tags. Such methods are commonly used by skilled practitioners.

Using appropriate amino acid sequence information, synthetic SCA-2 proteins of the present invention may be prepared by various synthetic methods of peptide synthesis via condensation of one or more amino acid residues, in accordance with conventional peptide synthesis methods. Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art.

Hence, in one particular embodiment, the present invention provides novel compositions containing an SCA-2 therapeutic element, specifically an SCA-2 polypeptide, protein or protein fragment, which may act as a regulator of normal body weight, and may be administered for the prevention of abnormal accumulation of body fat in individuals, in particular those suffering from obesity.

In particular the SCA-2 protein compositions and methods of the present invention may be used for the reversal or prevention of stress-induced obesity in predisposed individuals. Specifically, the compositions and methods of the present invention may be useful in the prevention or reversal of obesity brought about by a high-fat diet.

According to one specific aspect, this invention depends on an isolated polypeptide encoding an SCA-2 protein. Preferably, the polypeptide comprises the amino acid sequence of: SEQ ID NO:2, with up to 30% conservative substitutions. In a particular aspect, the invention relates to compositions and methods for using such polypeptides for treating obesity, by administering an SCA-2 polypeptide, in a pharmaceutically acceptable and appropriate delivery vehicle, to prevent, reverse or inhibit obesity.

Vectors, Host Cells, and Expression

In one particular embodiment the present invention relates to vectors that comprise SCA-2 therapeutic related genetic elements, such as a polynucleotide or polynucleotides of the present invention and to the production of polypeptides and proteins of the invention by recombinant techniques both in vitro and in vivo, as well as ex vivo procedures. Cell-free translation systems can also be employed to produce such proteins using RNAs derived from the DNA constructs of the present invention.

Host cells can be genetically engineered to incorporate expression systems or portions thereof for polynucleotides of the present invention. In accordance with the methods of the present invention, host cells may also be obtained from the subject by procedures well known in the medical research arts. Introduction of polynucleotides into host cells can then be effected by methods described in many standard laboratory manuals, such as Davis et al., Basic Methods In Molecular Biology (1986) and Sambrook et al., Molecular Cloning: A Laboratory Manual, 2nd Ed., Cold Spring Harbor Laboratory Press, Cold Spring Harbor, N.Y. (1989). These methods include calcium phosphate transfection, DEAE-dextran mediated transfection, microinjection, cationic lipid-mediated transfection, lipofectamine transfection, electroporation, transduction, scrape loading, ballistic introduction or infection.

Representative examples of appropriate hosts for in vitro procedures include bacterial cells, such as Streptococci, Staphylococci, E. coli, Streptomyces, Lactobacillus, Bacillus cells; fungal cells, such as non-pathogenic yeast cells; and animal cells such as CHO, COS, HeLa, C127, 3T3, BHK, HEK 293 and Bowes melanoma cells. The selection of an appropriate host is deemed to be within the scope of those skilled in the art from the teachings herein.

More particularly, the present invention also includes recombinant constructs comprising an SCA-2 DNA, cDNA or RNA sequence as well as compliment nucleotide sequences, i.e., for triplexing duplex DNA, and antisense polynucleotide sequences. The construct comprises a vector, such as a plasmid or viral vector, into which the clone has been inserted, in a forward or reverse orientation. In a preferred aspect of this embodiment, the construct further comprises regulatory sequences, including, for example, a promoter, operably linked to the genetic sequence, and may include a suitable origin of replication or termination sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX 174, pbluescript SK, pbsks, pNH8A, pNH 16a, pNHI8A, pNH46A (Stratagene); ptrc99a, pKK223-3, pKK233-3, pDR540, pRIT5 (Pharmacia); Eukaryotic: pWLNEO, pSV2CAT, pOG44, pXTI, pSG (Stratagene) pSVK3, pBPV, pMSG, pSVL (Pharmacia). However, any other plasmid or vector may be used as long as they are replicable and viable in the host.

In addition, a complete mammalian transcription unit and a selectable marker can be inserted into a prokaryotic plasmid for use in in vivo procedures. The resulting vector is then amplified in bacteria before being transfected into cultured mammalian cells, or delivered directly to the subject with an acceptable biological carrier as described below. Examples of vectors of this type include pTK2, pHyg, pRSVneo, pREP4 or BSRα. Hence, these plasmids, constructs and vectors may be used in both in vivo and ex vivo procedures. Ex vivo procedures involve the removal of a host cell from the subject, recombinant manipulation of the cell (i.e., transformation, transduction or transfection with a suitable SCA-2 expression system vector), and the re-delivery of the cell back into its host environment.

A wide variety of recombinant plasmids and delivery methods may be engineered to express the SCA-2 protein and used for delivery of SCA-2 to a cell. These include the use of naked DNA and SCA-2 plasmids to directly transfer genetic material into a cell; formulations of trapped liposomes encoding a therapeutic SCA-2 genetic element or in proteoliposomes that contain other viral envelope receptor proteins; and SCA-2 DNA coupled to a polylysine glycoprotein carrier complex. Hence methods for the delivery of nucleotide sequences to cells are well known in the recombinant arts. Such methods for in vitro delivery, further include, but are not limited to: microinjection, calcium phosphatase, liposomes, lipofectamine transfection and electroporation.

Genetic material, such as the nucleotides of the present invention, may be delivered to cells, in vivo, using various different plasmid based delivery platforms, including but not limited to recombinant adenoviruses (such as that described in U.S. Pat. No. 6,069,134 incorporated by reference herein), adeno-associated viruses (such as those described by U.S. Pat. No. 5,139,941 incorporated by reference herein), Herpes Simplex Virus (U.S. Pat. No. 5,288,641, incorporated by reference herein), cytomegalovirus, lentiviral, and overall, retroviral gene delivery systems, well known and practiced with in the art.

Techniques for preparing replication defective, infective viruses are well known in the art, as exemplified by Ghosh-Choudhury & Graham (EMBO Journal Vol 6, pp.1733-1739, 1987), incorporated by reference herein. These systems typically include a plasmid vector including a promoter sequence (such as CMV early promoter) linked to the nucleotide coding the gene of interest (inserted into an appropriate gene insertion site; i.e., an IRES site), as well as a terminating signal (such as a Poly-A tail i.e., BGH), and the appropriate mutations so as to make the delivery vehicle replication defective (e.g., Psi sequence deletions) and safe for therapeutic uses. The construction of the appropriate elements in a vector system containing the nucleotides of the present invention is well within the skills of one versed in the recombinant arts.

A great variety of vector and/or expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia, viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate SCA-2 therapeutic nucleotide sequence may be inserted into an expression system by any of a variety of well-known and routine techniques, such as, for example, those set forth in Sambrook et al., Molecular Cloning, A Laboratory Manual (supra).

Promoter regions can be selected from any desired gene using CAT (chloramphenicol acetyl transferase) vectors or other vectors with selectable markers. Two appropriate vectors are pKK232-8 and pCM7. Particular named bacterial promoters include 1acI, 1acZ, T3, T7, gpt, lambda PR, PL and trp. Eukaryotic promoters include CMV immediate early, HSV thymidine kinase, early and late SV40, LTRs from retrovirus, and mouse metallothionein-1. Selection of the appropriate vector and promoter is well within the level of ordinary skill in the art.

The therapeutic SCA-2 genetic elements of the present invention, such as DNA, cDNA, or RNA may be administered by use of biologically compatible carriers or excipients. Pharmaceutically acceptable carriers for therapeutic use are well known in the pharmaceutical arts, and are described, for example, in Remington's Pharmaceutical Sciences (A. P. Gennaro, ed.; Mack, 1985). For example, sterile saline or phosphate-buffered saline at physiological pH may be used. Preservatives, stabilizers, dyes, and even flavoring agents may be provided in the pharmaceutical composition. For example, sodium benzoate, sorbic acid, and esters of p-hydroxybenzoic acid may be added as preservatives. Antioxidants and suspending agents may also be used.

The above-described constructs, plasmids and vectors are useful in gene therapy procedures. Successful gene therapy generally requires the integration of a gene capable of correcting the genetic disorder into the host genome, where it would co-exist and replicate with the host DNA and be expressed at a level to compensate for the defective gene. Ideally, the disease would be cured by one or a few treatments, with no serious side effects. There are several approaches to gene therapy proposed.

As described above, basic transfection methods exist in which DNA containing the gene of interest is introduced into cells non-biologically, for example, by permeabilizing the cell membrane physically or chemically. Liposomes or protein conjugates formed with certain lipids and amphophilic peptides can be used for transfection. (Stewart et al.,J Clin Oncol. 1998 Feb.;16(2):683-91); Torchilin et al., Proc Natl Acad Sci U S A. Jul. 17, 2001 ;98(15):8786-91, each incorporated herein by reference.) This approach is particularly effective in ex vivo procedures involving cells which can be temporarily removed from the body and can tolerate the structural manipulation of the treatment.

A basic transduction approach capitalizes on the natural ability of viruses to enter cells, bringing their own genetic material with them. For example, retroviruses have promise as gene delivery vectors due to their ability to integrate their genes into the host genome, transferring a large amount of foreign genetic material, infecting a broad spectrum of species and cell types and of being packaged in special cell-lines (Miller, Curr. Top. Microbiol. Immunol. 158, 1-24, 1992, incorporated herein by reference).

A third method uses other viruses, such as adenovirus, herpes simplex virues (HSV), cytomegalovirus (CMV), and adeno-associated virus (AAV), which are engineered to serve as vectors for gene transfer. Although some viruses that can accept foreign genetic material are limited in the number of nucleotides they can accommodate and in the range of cells they infect, these viruses have been demonstrated to successfully effect gene expression. For example, adenovirus gene transfer systems may be used. Such a system is based upon recombinant, engineered adenovirus which is rendered replication-incompetent by deletion of a portion of its genome, such as E1, and yet still retains its competency for infection. Relatively large foreign proteins can be expressed when additional deletions are made in the adenovirus genome. For example, adenoviruses deleted in both E1 and E3 regions are capable of carrying up to 10 Kb of foreign DNA and can be grown to high titers in 293 cells (Stratford-Perricaudet and Perricaudet, Hum. Gene. Ther., 1:241-256,1991). Surprisingly persistent expression of transgenes following adenoviral infection has also been reported.

Hence, in one particular aspect the invention features a recombinant SCA-2 DNA or RNA molecule comprising a vector having an insert that includes part or all of an SCA-2 polynucleotide sequence. The invention also features a vector containing an SCA-2 polypeptide. Pharmaceutical compositions containing a biologically effective amount of the SCA-2 polynucleotide, antisense sequence, protein and/or protein fragments with acceptable carriers are also provided.

Specifically, in a particular embodiment, the invention relates to the administration of vectors for the delivery of an SCA-2 therapeutic element to a cell for the treatment of abnormal accumulation of body fat, wherein the vector contains an expression cassette encoding the SCA-2 therapeutic. The SCA-2 therapeutic can be an SCA-2 polynucleotide, an SCA-2 protein, or an SCA-2 protein fragment. The expression cassette may contain one or more of the following elements: a host cell origin of replication, a suitable promoter operably linked to a heterologous genetic element, an internal ribosome entry site, splice donor site, splice acceptor site, a suitable enhancer, PPT track, heterologous genetic element, a reporter gene, and/or an appropriate termination sequence. One or more of these vectors, containing an SCA-2 therapeutic, may be introduced into an appropriate cell by a variety of means, including in vivo, in vitro or ex vivo transduction or transfection using an appropriate expression system.

Delivery/Administration of SCA-2 Therapeutics

The pharmaceutical compositions of the present invention may be formulated and used as tablets, capsules, or elixirs for oral administration; suppositories for rectal or vaginal administration; sterile solutions and suspensions for parenteral administration; creams, lotions, or gels for topical administration; aerosols or insufflations for intratracheobronchial administration; and the like. Preparations of such formulations are well known to those skilled in the pharmaceutical arts. The dosage and method of administration can be tailored to achieve optimal efficacy and will depend on factors that those skilled in the medical arts will recognize.

When administration is to be parenteral, such as subcutaneous, intramuscular, or intravenous on a daily basis, injectable pharmaceuticals may be prepared in conventional forms, either as liquid solutions or suspensions; solid forms suitable for solution or suspension in liquid prior to injection; or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g. liposomes) may be utilized.

Hence, in another preferred embodiment the present invention is directed to a novel pharmaceutical composition that includes a biologically acceptable carrier along with an effective amount of a therapeutic SCA-2 genetic element such as a SCA-2 DNA, cDNA, RNA or protein for the treatment and/or prevention of obesity. The pharmaceutical composition includes a SCA-2 sequence substantially identical to SEQ ID No. 1 and/or a protein encoded by an amino acid sequence substantially identical to the sequence of SEQ ID No. 2.

The methods for the prophylaxis and treatment of obesity are also provided. These methods involve administering to a subject a pharmaceutical composition that includes an effective amount of a therapeutic SCA-2 genetic element, which may include an SCA-2 protein or a nucleotide sequence coding for the SCA-2 protein. These may be delivered by suitable means, as described above, including the use of vectors and or acceptable biological carriers.

For administration, the therapeutic agent will generally be mixed, prior to administration, with a non-toxic, pharmaceutically acceptable carrier substance. Usually, this will be an aqueous solution, such as normal saline or phosphate-buffered saline (PBS), Ringer's solution, lactate-Ringer's solution, or any isotonic physiologically acceptable solution for administration by the means. Preferably, the solution is sterile and pyrogen-free, and is manufactured and packaged under current Good Manufacturing Processes (GMP's), as approved by the FDA. The clinician of ordinary skill is familiar with appropriate ranges for pH, tonicity, and additives or preservatives when formulating pharmaceutical compositions for administration by intravascular injection, intrathecal injection, or by other routes. In addition to additives for adjusting pH or tonicity, the therapeutic agent may be stabilized against aggregation and polymerization with amino acids and non-ionic detergents, polysorbate, and polyethylene glycol.

Optionally, additional stabilizers may include various physiologically acceptable carbohydrates and salts. Also, polyvinylpyrrolidone may be added in addition to the amino acid. The compositions of the invention may be administered using any medically appropriate procedure, e.g., intravascular (intravenous, intraarterial, intracapillary) administration, injection into muscle, neural, or adipose tissue. Intravascular injection may be by intravenous or intraarterial injection.

The effective amount of the therapeutic composition to be given to a particular patient will depend on a variety of factors, several of which will be different from patient to patient. A competent clinician will be able to determine an effective amount of a therapeutic composition to administer to a patient to reverse obesity. Dosage of the therapeutic will depend on the type of treatment, route of administration, the nature of the therapeutics, sensitivity of the cell to the therapeutics, etc. Utilizing LD₅₀ animal data, and other information available for the administration of such compositions, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions, which are rapidly cleared from the body, may be administered at higher doses, or in repeated doses, in order to maintain a therapeutic concentration. Utilizing ordinary skill, the competent clinician will be able to optimize the dosage of a particular therapeutic composition in the course of routine clinical trials.

Typically the dosage will be 0.001 to 100 milligrams of SCA-2 therapeutic per kilogram subject body weight. Doses in the range of 0.01 to 1 mg per kilogram of patient body weight may be utilized for a therapeutic composition that is administered. The SCA-2 therapeutic can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, moieties that do not provoke HAMA or other immune responses are preferred.

The foregoing is intended to be illustrative of the embodiments of the present invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are herein incorporated by reference to the same extent as if each individual publication or patent application was specifically and individually indicated to be incorporated by reference.

### Examples

The following description sets forth the general procedures involved in practicing the present invention. To the extent that specific materials are mentioned, it is merely for purposes of illustration and is not intended to limit the invention. Unless other-wise specified, general cloning procedures, such as those set forth in Sambrook et al., Molecular Cloning, supra or Ausubel et al. (eds) Current Protocols in Molecular Biology, John Wiley & Sons (2000) are used.

### Example I

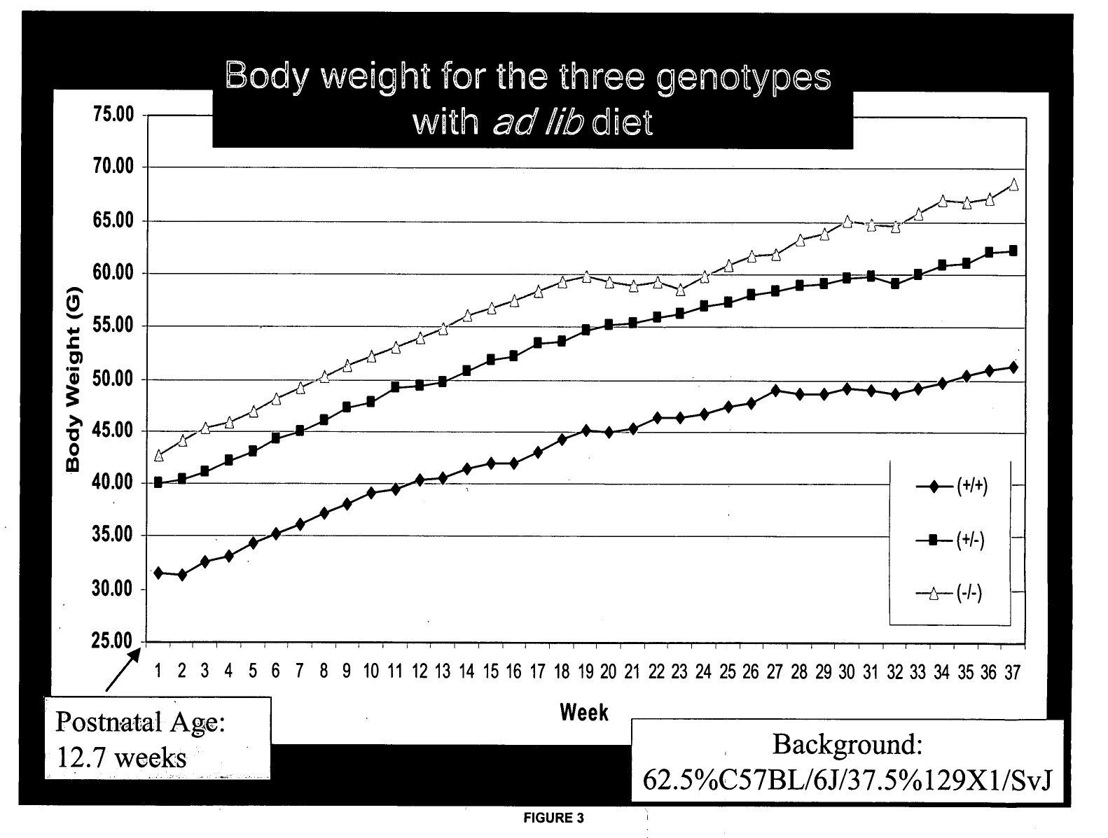

Administration of SCA-2 therapeutic polypeptides: SCA-2 therapeutic polypeptides are produced either by recombinant means or by chemical synthesis and their effects on body weight and food intake in wild-type, SCA-2 knockout, and hemizygous mice is subsequently assessed. When administered parenterally for 28 consecutive days, the SCA-2 polypeptides are expected to significantly restrict weight gain and food intake when compared to saline-injected control. Initial dosages at 0.5, 1 and 2 mg per day will be administered to different groups of each genotype to determine the optimal dosis. Subsequent dosage adjustments will be determined based on the data obtained. Weight gain or loss will be measured weekly throughout the duration of the treatment. The effect of SCA-2 therapeutic polypeptides on food intake will be measured on a daily basis. All data will be charted as in FIGS. 3 - 6 and subjected to statistical analysis.

### Example II

Administration of SCA-2 polynucleotides: The three genotypes of 8 week old mice (SCA-2 knockout, hemizygous, and wild type) are fed milled chow for several days prior to the treatment regimen. During this period, the mice are weighed and bled for determination of plasma levels of glucose and insulin. Injections are started 6-8 days after the initiation of base line measurements and after food consumption is determined to a fairly constant level for each genotype. Mice are housed 5 per cage and fed milled chow in feed cups with lids to prevent spillage and allow accurate measurements of food consumption. Daily food consumption is measured at the same time each day. On the day of injection, food consumption, body weight, and a baseline blood sample are taken prior to injection. Hematocrit, glucose, and insulin levels are measured and recorded. Mice are injected intravascularly at dosages ranging from 10⁸ pfu SCA-2 expressing vector per gram weight to 10¹⁰ pfu per gram weight in the case of viral vectors. The control groups receive empty vector and saline, respectively.

SEQUENCE LISTING

SEQ ID NO: 1

```
      1 acccccgaga aagcaaccca gcgcgccgcc cgctcctcac gtgtccctcc cggccccggg
    61 gccacctcac gttctgcttc cgtctgaccc ctccgacttc cggtaaagag tccctatccg
  121 cacctccgct cccacccggc gcctcggcgc gcccgccctc cgatgcgctc agcggccgca
  181 gctcctcgga gtcccgcggt ggccaccgag tctcgccgct tcgccgcagc caggtggccc
  241 gggtggcgct cgctccagcg gccggcgcgg cggagcgggc ggggcggcgg tggcgcggcc
  301 ccgggaccgt atccctccgc cgcccctccc ccgcccggcc ccggcccccc tccctcccgg
  361 cagagctcgc ctccctccgc ctcagactgt tttggtagca acggcaacgg cggcggcgcg
  421 tttcggcccg gctcccggcg gctccttggt ctcggcgggc ctccccgccc cttcgtcgtc
  481 gtccttctcc ccctcgccag cccgggcgcc cctccggccg cgccaacccg cgcctccccg
  541 ctcggcgccc gtgcgtcccc gccgcgttcc ggcgtctcct tggcgcgccc ggctcccggc
  601 tgtccccgcc cggcgtgcga gccggtgtat gggcccctca ccatgtcgct gaagccccag
  661 cagcagcagc agcagcagca gcaacagcag cagcagcaac agcagcagca gcagcagcag
  721 cagccgccgc ccgcggctgc caatgtccgc aagcccggcg gcagcggcct tctagcgtcg
  781 cccgccgccg cgccttcgcc gtcctcgtcc tcggtctcct cgtcctcggc cacggctccc
  841 tcctcggtgg tcgcggcgac ctccggcggc gggaggcccg gcctgggcag aggtcgaaac
  901 agtaacaaag gactgcctca gtctacgatt tcttttgatg gaatctatgc aaatatgagg
  961 atggttcata tacttacatc agttgttggc tccaaatgtg aagtacaagt gaaaaatgga
1021 ggtatatatg aaggagtttt taaaacttac agtccgaagt gtgatttggt acttgatgcc
1081 gcacatgaga aaagtacaga atccagttcg gggccgaaac gtgaagaaat aatggagagt
1141 attttgttca aatgttcaga ctttgttgtg gtacagttta aagatatgga ctccagttat
1201 gcaaaaagag atgcttttac tgactctgct atcagtgcta aagtgaatgg cgaacacaaa
1261 gagaaggacc tggagccctg ggatgcaggt gaactcacag ccaatgagga acttgaggct
1321 ttggaaaatg acgtatctaa tggatgggat cccaatgata tgtttcgata taatgaagaa
1381 aattatggtg tagtgtctac gtatgatagc agtttatctt cgtatacagt gcccttagaa
1441 agagataact cagaagaatt tttaaaacgg gaagcaaggg caaaccagtt agcagaagaa
1501 attgagtcaa gtgcccagta caaagctcga gtggccctgg aaaatgatga taggagtgag
1561 gaagaaaaat acacagcagt tcagagaaat tccagtgaac gtgaggggca cagcataaac
1621 actagggaaa ataaatatat tcctcctgga caaagaaata gagaagtcat atcctgggga
1681 agtgggagac agaattcacc gcgtatgggc cagcctggat cgggctccat gccatcaaga
1741 tccacttctc acacttcaga tttcaacccg aattctggtt cagaccaaag agtagttaat
1801 ggaggtgttc cctggccatc gccttgccca tctccttcct ctcgcccacc ttctcgctac
1861 cagtcaggtc ccaactctct tccacctcgg gcagccaccc ctacacggcc gccctccagg
1921 cccccctcgc ggccatccag acccccgtct cacccctctg ctcatggttc tccagctcct
1981 gtctctacta tgcctaaacg catgtcttca gaagggcctc caaggatgtc cccaaaggcc
2041 cagcgacatc ctcgaaatca cagagtttct gctgggaggg gttccatatc cagtggccta
2101 gaatttgtat cccacaaccc acccagtgaa gcagctactc ctccagtagc aaggaccagt
2161 ccctcggggg gaacgtggtc atcagtggtc agtggggttc caagattatc ccctaaaact
2221 catagaccca ggtctcccag acagaacagt attggaaata cccccagtgg gccagttctt
2281 gcttctcccc aagctggtat tattccaact gaagctgttg ccatgcctat tccagctgca
2341 tctcctacgc ctgctagtcc tgcatcgaac agagctgtta ccccttctag tgaggctaaa
2401 gattccaggc ttcaagatca gaggcagaac tctcctgcag ggaataaaga aaatattaaa
2461 cccaatgaaa catcacctag cttctcaaaa gctgaaaaca aaggtatatc accagttgtt
2521 tctgaacata gaaaacagat tgatgattta aagaaattta agaatgattt taggttacag
2581 ccaagttcta cttctgaatc tatggatcaa ctactaaaca aaaatagaga gggagaaaaa
2641 tcaagagatt tgatcaaaga caaaattgaa ccaagtgcta aggattcttt cattgaaaat
2701 agcagcagca actgtaccag tggcagcagc aagccgaata gccccagcat ttccccttca
2761 atacttagta acacggagca caagagggga cctgaggtca cttcccaagg ggttcagact
2821 tccagcccag catgtaaaca agagaaagac gataaggaag agaagaaaga cgcagctgag
2881 caagttagga aatcaacatt gaatcccaat gcaaaggagt tcaacccacg ttccttctct
2941 cagccaaagc cttctactac cccaacttca cctcggcctc aagcacaacc tagcccatct
3001 atggtgggtc atcaacagcc aactccagtt tatactcagc ctgtttgttt tgcaccaaat
3061 atgatgtatc cagtcccagt gagcccaggc gtgcaacctt tatacccaat acctatgacg
3121 cccatgccag tgaatcaagc caagacatat agagcagtac caaatatgcc ccaacagcgg
3181 caagaccagc atcatcagag tgccatgatg cacccagcgt cagcagcggg cccaccgatt
3241 gcagccaccc caccagctta ctccacgcaa tatgttgcct acagtcctca gcagttccca
3301 aatcagcccc ttgttcagca tgtgccacat tatcagtctc agcatcctca tgtctatagt
3361 cctgtaatac agggtaatgc tagaatgatg gcaccaccaa cacacgccca gcctggttta
3421 gtatcttctt cagcaactca gtacggggct catgagcaga cgcatgcgat gtatgcatgt
3481 cccaaattac catacaacaa ggagacaagc ccttctttct actttgccat ttccacgggc
3541 tcccttgctc agcagtatgc gcaccctaac gctaccctgc acccacatac tccacaccct
3601 cagccttcag ctacccccac tggacagcag caaagccaac atggtggaag tcatcctgca
3661 cccagtcctg ttcagcacca tcagcaccag gccgcccagg ctctccatct ggccagtcca
3721 cagcagcagt cagccattta ccacgcgggg cttgcgccaa ctccaccctc catgacacct
3781 gcctccaaca cgcagtcgcc acagaatagt ttcccagcag cacaacagac tgtctttacg
3841 atccatcctt ctcacgttca gccggcgtat accaacccac cccacatggc ccacgtacct
3901 caggctcatg tacagtcagg aatggttcct tctcatccaa ctgcccatgc gccaatgatg
3961 ctaatgacga cacagccacc cggcggtccc caggccgccc tcgctcaaag tgcactacag
4021 cccattccag tctcgacaac agcgcatttc ccctatatga cgcacccttc agtacaagcc
4081 caccaccaac agcagttgta aggctgccct ggaggaaccg aaaggccaaa ttccctcctc
4141 ccttctactg cttctaccaa ctggaagcac agaaaactag aatttcattt attttgtttt
4201 taaaatatat atgttgattt cttgtaacat ccaataggaa tgctaacagt tcacttgcag
4261 tggaagatac ttggaccgag tagaggcatt taggaacttg ggggctattc cataattcca
4321 tatgctgttt cagagtcccg caggtacccc agctctgctt gccgaaactg gaagttattt
4381 attttttaat aacccttgaa agtcatgaac acatcagcta gcaaaagaag taacaagagt
4441 gattcttgct gctattactg ctaaaaaaaa aaaaaaaaaa a
```

SEQ ID NO: 2

MRSAAAAPRSPAVATESRRFAAARWPGWRSLQRPARRSGRGGGG
AAPGPYPSAAPPPPGPGPPPSRQSSPPSASDCFGSNGNGGGAFRPGSRRLLGLGGPPR
PFVVVLLPLASPGAPPAAPTRASPLGARASPPRSGVSLARPAPGCPRPACEPVYGPLT
MSLKPQQQQQQQQQQQQQQQQQQQQQQPPPAAANVRKPGGSGLLASPAAAPSPSSSSV
SSSSATAPSSVVAATSGGGRPGLGRGRNSNKGLPQSTISFDGIYANMRNVHILTSVVG
SKCEVQVKNGGIYEGVFKTYSPKCDLVLDAAHEKSTESSSGPKREEIMESILFKCSDF
VVVQFKDMDSSYAKRDAFTDSAISAKVNGEHKEKDLEPWDAGELTANEELEALENDVS
NGWDPNDMFRYNEENYGVVSTYDSSLSSYTVPLERDNSEEFLKREARANQLAEETESS
AQYKARVALENDDRSEEEKYTAVQRNSSEREGHSINTRENKYIPPGQRNREVISWGSG
RQNSPRMGQPGSGSMPSRSTSHTSDFNPNSGSDQRVVNGGVPWPSPCPSPSSRPPSRY
QSGPNSLPPRAATPTRPPSRPPSRPSRPPSHPSAHGSPAPVSTMPKRNSSEGPPRISP
KAQRHPRNHRVSAGRGSISSGLEFVSHNPPSEAATPPVARTSPSGGTWSSVVSGVPRL
SPKTHRPRSPRQNSIGNTPSGPVLASPQAGIIPTEAVAMPIPAASPTPASPASNPAVT
PSSEAKDSRLQDQRQNSPAGNKENIKPNETSPSFSKAENKGISPVVSEHRKQIDDLKK
FKNDFRLQPSSTSESMDQLLNKNREGEKSRDLIKDKIEPSAKDSFIENSSSNCTSGSS
KPNSPSISPSILSNTEHKRGPEVTSQGVQTSSPACKQEKDDKEEKKDAAEQVRKSTLN
PNAKEFNPRSFSQPKPSTTPTSPRPQAQPSPSMVGHQQPTPVYTQPVCFAPNMMYPVP
VSPGVQPLYPIPMTPMPVNQAKTYRAVPNMPQQRQDQHHQSAMMHPASAAGPPIAATP
PAYSTQYVAYSPQQFPNQPLVQHVPHYQSQHPHVYSPVIQGNARMMAPPTHAQPGLVS
SSATQYGAHEQTHAMYACPKLPYNKETSPSFYFAISTGSLAQQYAHPNATLHPHTPHP
QPSATPTGQQQSQHGGSHPAPSPVQHHQHQAAQALHLASPQQQSAIYHAGLAPTPPSM
TPASNTQSPQNSFPAAQQTVFTIHPSHVQPAYTNPPHMAHVPQAHVQSGMVPSHPTAH
APMMLMTTQPPGGPQAALAQSALQPIPVSTTAHFPYMTHPSVQAHHQQQL

1. A vector suitable for use in a human comprising a polynucleotide, wherein said polynucleotide encodes an SCA-2 polypeptide.

2. The vector of claim 1, wherein said polynucleotide sequence encoding the SCA-2 polypeptide is SEQ ID NO:1.

3. The vector of claim 1, wherein the polynucleotide sequence has at least 70% identity to SEQ ID NO:1, said identity being calculated over the entire length of SEQ ID NO:1.

4. The vector of claim 1, wherein the polynucleotide sequence is identical to SEQ ID No:1.

5. The vector of claim 1, wherein the SCA-2 polypeptide comprises an amino acid sequence of SEQ ID NO:2.

6. The vector of claim 5, wherein the SCA-2 polypeptide comprises an amino acid sequence that has at least 70% identity to SEQ ID NO:2, said identity being calculated over the entire length of SEQ ID NO:2.

7. A pharmaceutical composition suitable for use in a human comprising a biologically effective amount of an SCA-2 polynucleotide and an acceptable carrier.

8. The composition of claim 7, wherein the SCA-2 polynucleotide sequence is substantially similar to SEQ ID NO:1.

9. A pharmaceutical composition suitable for use in a human comprising a biologically effective amount of an SCA-2 polypeptide and an acceptable carrier.

10. The composition of claim 9, wherein the SCA-2 polypeptide sequence is substantially similar to SEQ ID NO:2.

11. A method of treating obesity comprising the administration of a pharmaceutical composition comprising a biologically effective amount of an SCA-2 polynucleotide and an acceptable carrier.

12. The method of claim 11, wherein the obesity comprises stress-induced obesity.

13. A method of treating obesity comprising the administration of a pharmaceutical composition comprising a biologically effective amount of an SCA-2 polypeptide and an acceptable carrier.

14. The method of claim 13, wherein the obesity comprises stress-induced obesity.

15. A method of treating the abnormal accumulation of body fat comprising the administration of a pharmaceutical composition comprising a biologically effective amount of a polynucleotide coding for the antisense sequence to SEQ. ID. No. 1, and an acceptable carrier.

16. A vector for the delivery of an SCA-2 therapeutic element to a human for the treatment of obesity wherein the vector comprises an expression cassette encoding the SCA-2 therapeutic.

17. The vector of claim 16, wherein the SCA-2 therapeutic is selected from the group consisting of an SCA-2 polynucleotide, an SCA-2 protein, and an SCA-2 protein fragment.

18. The vector of claim 17, wherein the expression cassette comprises one or more elements selected from the group consisting of a host cell origin of replication, suitable promoter operably linked to a heterologous genetic element, internal ribosome entry site, splice donor site, splice acceptor site, suitable enhancer, PPT track, heterologous genetic element, a reporter gene, and an appropriate termination sequence.

19. The vector of claim 17 wherein the vector is selected from the group consisting of: retrovirus, lentivirus, adenovirus, herpes simplex viruses (HSV), cytomegalovirus (CMV), and adeno-associated virus (AAV).

20. A method for introducing an SCA-2 therapeutic into a human for the treatment of obesity, comprising transducing the cell with the vector of claim 19.

21. The method of claim 20, wherein the transduction occurs in vivo.

22. The method of claim 20, wherein the transduction occurs ex vivo.

23. The method of claim 20, wherein the cell is selected from the group consisting of muscle cells and adipocytes.

24. The cell of claim 21, wherein the cell comprises a neural cell.

25. A method for introducing an SCA-2 therapeutic into a human for the treatment of obesity, comprising transfecting a cell with a plasmid comprising an expression cassette encoding the SCA-2 therapeutic.

26. The method of claim 25, wherein the SCA-2 therapeutic is selected from the group consisting of an SCA-2 polynucleotide, an SCA-2 protein and an SCA-2 protein fragment.

27. The method of claim 25, wherein said transfection is carried out by a procedure selected from the group consisting of calcium phosphate transfection, DEAE-dextran mediated transfection, transvection, microinjection, cationic lipid-mediated transfection, electroporation, scrape loading, ballistic introduction or infection, use of a gene gun, liposome and lipofectamine transfection.

28. The method of claim 27, wherein the transfection occurs in vivo.

29. The method of claim 27, wherein the transfection occurs in vitro.

30. The method of claim 27, wherein the cell is selected from the group consisting of muscle, neural and adipose cells.

31. The method of claim 30, wherein the transfection takes place as part of an ex vivo procedure.


SEQUENCE LISTING (2 sequences)

SEQ ID NO: 1 - DNA, 4481 bp, Homo sapiens
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcgtccttctccccctcgccagcccgggcgcccctccggccgcgccaacccgcgcctccccgctcggcgcccgtgcgtccccgccgcgttccggcgtctccttggcgcgcccggctcccggctgtccccgcccggcgtgcgagccggtgtatgggcccctcaccatgtcgctgaagccccagcagcagcagcagcagcagcagcaacagcagcagcagcaacagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaa

SEQ ID NO: 2 - protein, 1312 aa, Homo sapiens
MRSAAAAPRSPAVATESRRFAAARWPGWRSLQRPARRSGRGGGGAAPGPYPSAAPPPPGPGPPPSRQSSPPSASDCFGSNGNGGGAFRPGSRRLLGLGGPPRPFVVVLLPLASPGAPPAAPTRASPLGARASPPRSGVSLARPAPGCPRPACEPVYGPLTMSLKPQQQQQQQQQQQQQQQQQQQQQQPPPAAANVRKPGGSGLLASPAAAPSPSSSSVSSSSATAPSSVVAATSGGGRPGLGRGRNSNKGLPQSTISFDGIYANMRMVHILTSVVGSKCEVQVKNGGIYEGVFKTYSPKCDLVLDAAHEKSTESSSGPKREEIMESILFKCSDFVVVQFKDMDSSYAKRDAFTDSAISAKVNGEHKEKDLEPWDAGELTANEELEALENDVSNGWDPNDMFRYNEENYGVVSTYDSSLSSYTVPLERDNSEEFLKREARANQLAEEIESSAQYKARVALENDDRSEEEKYTAVQRNSSEREGHSINTRENKYIPPGQRNREVISWGSGRQNSPRMGQPGSGSMPSRSTSHTSDFNPNSGSDQRVVNGGVPWPSPCPSPSSRPPSRYQSGPNSLPPRAATPTRPPSRPPSRPSRPPSHPSAHGSPAPVSTMPKRMSSEGPPRMSPKAQRHPRNHRVSAGRGSISSGLEFVSHNPPSEAATPPVARTSPSGGTWSSVVSGVPRLSPKTHRPRSPRQNSIGNTPSGPVLASPQAGIIPTEAVAMPIPAASPTPASPASNRAVTPSSEAKDSRLQDQRQNSPAGNKENIKPNETSPSFSKAENKGISPVVSEHRKQIDDLKKFKNDFRLQPSSTSESMDQLLNKNREGEKSRDLIKDKIEPSAKDSFIENSSSNCTSGSSKPNSPSISPSILSNTEHKRGPEVTSQGVQTSSPACKQEKDDKEEKKDAAEQVRKSTLNPNAKEFNPRSFSQPKPSTTPTSPRPQAQPSPSMVGHQQPTPVYTQPVCFAPNMMYPVPVSPGVQPLYPIPMTPMPVNQAKTYRAVPNMPQQRQDQHHQSAMMHPASAAGPPIAATPPAYSTQYVAYSPQQFPNQPLVQHVPHYQSQHPHVYSPVIQGNARMMAPPTHAQPGLVSSSATQYGAHEQTHAMYACPKLPYNKETSPSFYFAISTGSLAQQYAHPNATLHPHTPHPQPSATPTGQQQSQHGGSHPAPSPVQHHQHQAAQALHLASPQQQSAIYHAGLAPTPPSMTPASNTQSPQNSFPAAQQTVFTIHPSHVQPAYTNPPHMAHVPQAHVQSGMVPSHPTAHAPMMLMTTQPPGGPQAALAQSALQPIPVSTTAHFPYMTHPSVQAHHQQQL